(19) United States
(12) Patent Application Publication
(10) Pub. No.: US 2009/0076416 A1
(43) Pub. Date: Mar. 19, 2009

(54) Guidewire with Adjustable Core

(75) Inventors: Kevin Treacy, Athenry (IE); Sean Martin, Tuam (IE); Grace Kelly, Boharmore (IE); Paul Mannion, Corrandulla (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/856,376

(22) Filed: Sep. 17, 2007

(51) Int. Cl. A61M 25/09

(52) U.S. Cl. 600/585; 600/434

(57) ABSTRACT

A movable-core guidewire including a proximal portion and a distal portion. The proximal portion includes a proximal hollow tube. The distal portion includes a coil coupled to the proximal hollow tube and an elastomeric distal tube disposed within the coil. A core wire is disposed within the hypotube. The core wire is movable between a distal position wherein a portion of the core wire is disposed in the distal tube and a proximal portion wherein the core wire is removed from the distal tube. A method is also disclosed for advancing the guidewire to the treatment site.

### FIELD OF THE INVENTION

The present disclosure relates to medical guidewires, and more particularly, a guidewire with a movable or adjustable core. Such a guidewire can be used in interventional cardiovascular procedures such as balloon angioplasty, atherectomy, stent implantation procedures, or radiology procedures.

### BACKGROUND OF THE INVENTION

One of the therapeutic procedures applicable to the present invention is known as percutaneous transluminal coronary angioplasty (PTCA). This procedure can be used, for example, to treat arterial build-up of cholesterol fats or atherosclerotic plaque in blood vessels of a patient. Typically, a guidewire is steered through the vascular system to the treatment site and a balloon dilatation catheter is advanced over, or together with the guidewire. A guiding catheter may be utilized to provide a conduit for directing the guidewire and/or dilatation catheter from a minimally-invasive entry site to a location near the treatment site. The balloon at the distal end of the catheter is inflated causing the site of the stenosis to widen. The original catheter can then be withdrawn and a catheter of a different size or another device such as an atherectomy device can be inserted.

The major considerations in guidewire design include steerability, flexibility, medial stiffness or support, bending in transition areas, tip formability and radiopacity. In a typical guidewire construction a stainless steel core wire has a platinum spring coil disposed around a tapered distal end of the core wire. A blunt tip is typically welded to the distal end of the guidewire to reduce trauma to the blood vessel.

Conventional guidewire designs are a trade-off between flexibility and steerability/stiffness. A flexible guidewire is needed to track through the tortuous vasculature. However, a stiff guidewire is often needed to cross a stenosis at the treatment site, or to guide a relatively stiff interventional device such as a compressed stent carried by a catheter into a curved region. In some instances, one guidewire needs to be exchanged for another guidewire with different properties to successfully cross the treatment site. Exchanging guidewires is time consuming and, in the case of so-called rapid exchange or single-operator catheters, exchanging guidewires is impossible or impractical. Accordingly, what is needed is a guidewire having a distal portion that can be more flexible when tracking through the vasculature and stiffer when needed to cross a stenosis or guide a stent across a stenosis.

### BRIEF SUMMARY OF THE INVENTION

The present disclosure is a guidewire and method of advancing the guidewire to a treatment site within a vessel. The guidewire includes a proximal portion and a distal portion. The proximal portion includes a proximal hollow tube. The distal portion includes a distal tube made of elastomeric material, located inside a coil. The coil is disposed around the distal tube. A core wire is disposed within the hypotube and is movable between a proximal position and a distal position. When the core wire is in the proximal position, the distal portion of the guidewire is more flexible than when the core wire is in the distal position.

The method of advancing the guidewire to a treatment site includes inserting the guidewire into the vessel and advancing the guidewire with the core wire in the proximal position. The core wire is moved distally into the distal position in order to increase bending stiffness in the distal portion of the guidewire.

### BRIEF DESCRIPTION OF DRAWINGS

The foregoing and other features and advantages of the disclosure will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 illustrates a side view of a guidewire in accordance with an embodiment of the present disclosure.

FIG. 2 illustrates a longitudinal cross-section of the guidewire of FIG. 1 with the core wire in a distal position.

FIG. 3 illustrates a longitudinal cross-section of the guidewire of FIG. 1 with the core wire in a proximal position.

FIG. 4 illustrates a cross-sectional view taken along line A-A of FIG. 2.

FIG. 5 illustrates a longitudinal cross-section of another embodiment of a guidewire in accordance with the invention, shown with the core wire in a distal position.

FIG. 6 illustrates a longitudinal cross-section of the guidewire of FIG. 5 with the core wire in a proximal position.

FIG. 7 illustrates a cross-sectional view taken along line B-B of FIG. 5.

### DETAILED DESCRIPTION OF THE INVENTION

Specific embodiments of the present disclosure are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

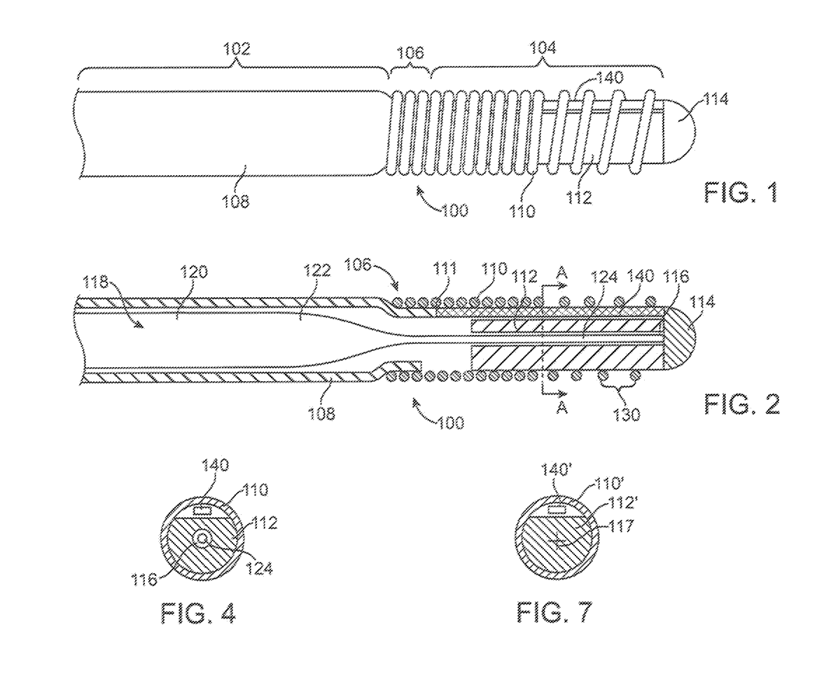

FIG. 1 of the present disclosure illustrates a side view of a guidewire 100 including a proximal section 102, a transition section 106, and a distal section 104. FIG. 1 does not show all of guidewire 100 and, as would be recognized by one of ordinary skill in the art, proximal section 102 extends proximally to achieve the desired length of guidewire 100. For example, guidewire 100 may be 135-310 cm long, with proximal section 102 being 95-270 cm long and distal section 104 being 10-40 cm long.

As illustrated in FIGS. 1 and 2, proximal section 102 comprises a hollow hypotube 108. Hypotube 108 may be made from relatively stiff materials, for example, stainless steel, or cobalt chromium alloy. Distal section 104 includes a coil 110, a flexible distal tube 112 disposed within coil 110, and a rounded tip 114. Tip 114 is coupled to the distal end of coil 110 and may be made of an adhesive, solder or welded metal. Coil 110 is helically mounted around flexible distal tube 112 and is coupled to a distal portion of hypotube 108 at transition region 106 by adhesive, soldering or welding. As shown in FIG. 2, the distal portion of hypotube 108 may be necked down or otherwise reduced in outer diameter at transition region 106 such that coil 110 may be coupled to hypotube 108 in a lap joint without increasing the profile of guidewire 100. Alternatively, the distal portion of hypotube 108 may remain unmodified and a proximal portion of coil 110 may be coupled to an interior surface of hypotube 108 at transition region 106. Other ways of coupling coil 110 to hypotube 108 would generally be recognized by those of ordinary skill in the art. Coil 110 may be fabricated from metal, such as stainless steel, tungsten, platinum or alloys of metals (platinum-tungsten, gold-iridium, or platinum-iridium, for example).

Coil 110 may be provided with a gap 130 between longitudinally adjacent turns along the full length or a portion of coil 110. Gap 130 may be relatively large to provide improved flexibility of distal portion 104 or may be relatively small for improved pushability, depending upon the application. Gap 130 may also vary from a proximal portion of distal portion 104 to a distal portion of distal portion 104. For example, gap 130 may increase toward the distal end of distal portion 104, as shown in FIGS. 1 and 2.

Distal tube 112 is bonded internally to tip 114 using an adhesive. Adhesives such as cyanoacrylate or epoxy may be used, although those skilled in the art would recognize that a number of biocompatible adhesives would be satisfactory. As shown in FIG. 2, distal tube 112 extends proximally from tip 114 towards proximal section 102. The length of distal tube 112 may be varied, typically from 1-40 cm., depending on the characteristics desired in the distal portion 104 of guidewire 100. For a more flexible distal portion 104, distal tube 112 will be shorter. For a more rigid distal portion 104, distal tube 112 will be longer and extend closer to proximal portion 102. Distal tube 112 further includes a bore 116. Bore 116 is sized and shaped to receive a distal section 124 of a movable core wire 118, as explained in more detail below.

Distal tube 112 may be made from elastomeric materials such as natural rubber, polyisoprene, butyl rubber (copolymer of isobutylene and isoprene, IIR), halogenated butyl rubbers (chloro butyl rubber: CIIR; bromo butyl rubber: BIIR), polybutadiene, styrene-butadiene rubber (copolymer of polystyrene and polybutadiene, SBR), nitrile rubber, (copolymer of polybutadiene and acrylonitrile, NBR), Bayer Inc's Therban® and ZEON Corp's Zetpol® hydrated nitrile rubbers (HNBR), Bayer Inc's Baypren® chloroprene rubber (CR), polychloroprene, neoprene, ethylene propylene rubber, ethylene propylene diene rubber, epichlorohydrin rubber (ECO), polyacrylic rubber (ACM, ABR), silicone rubber (SI, Q, VMQ), fluorosilicone rubber (FVMQ), fluoroelastomers, and perfluoroelastomers.

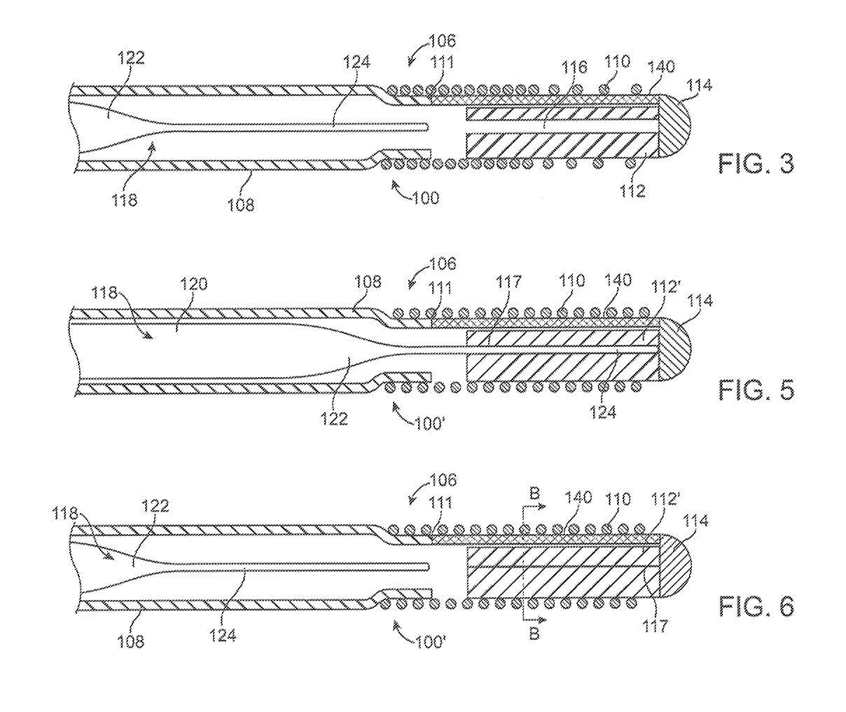

As illustrated in FIG. 2, guidewire 100 further includes movable core wire 118. Core wire 118 includes a proximal section 120, a distal section 124 having a smaller diameter than proximal section 120, and tapered or transition section 122 between proximal section 120 and distal section 124. Core wire 118 is movable within hypotube 108 and distal tube 112 from a distal or extended position shown in FIGS. 2 and 4 to a proximal or retracted position shown in FIG. 3. With core wire 118 in the distal position, distal portion 124 of core wire 118 is disposed within bore 116 of distal tube 112. Transition section 122 and proximal section 120 of core wire 118 may be disposed within hypotube 108. Alternatively, transition section 122 may extend distally from hypotube 108 into the interior of coil 110. With core wire 118 in the proximal position, distal portion 124 of core wire 118 is disposed proximally of distal tube 112, as shown in FIG. 3. When core wire 118 is in the distal position, distal portion 104 is more rigid than when core wire 118 is in the proximal position. Thus, core wire 118 may be in the proximal position when guidewire 100 needs to maneuver through tight turns of the vasculature and core wire 118 may be in the distal position when guidewire 100 needs to pass through, or to guide a relatively stiff catheter through a lesion in the vasculature. Those of ordinary skill in the art would recognize that core wire 118 may be disposed in several intermediate positions where core wire 118 is not completely inserted into distal tube 112, nor is core wire 118 completely removed from distal tube 112. In all possible positions of core wire 118 with respect to hypotube 108, coil 110, and distal tube 112, a proximal end portion of core wire 118 extends from the proximal end of hypotube 108 (not shown) for manual or mechanically-aided manipulation and control.

A forming ribbon may also be provided in distal section 104 of guidewire 100, as shown in FIGS. 1-5. Forming ribbon 140 may be made of stainless steel, cobalt chromium alloy or other suitable materials as would be understood by those of ordinary skill in the art. Forming ribbon 140 is coupled at its distal end to a distal portion of coil 110 and to tip 114. Forming ribbon 140 is coupled at its proximal end to a distal portion of hypotube 108 at junction 111. Forming ribbon 140 may be coupled at its ends by adhesive, solder or welds. Alternatively, forming ribbon 140 may be a unitary, slender extension of hypotube 108, formed, for example, by eccentrically removing material from a distal portion of hypotube 108.

Forming ribbon 140 serves a number of functions, including, but not limited to, holding the flexible distal tube 112 at a fixed position with respect to hypotube 108 while core wire 118 is pushed into bore 116, and transmitting rotation from hypotube 108 to distal tip 114 to aid in steering guidewire 100. It will be understood that relative movement between core wire 118 and distal tube 112 can be accomplished by moving either component while the other component is held steady, or by simultaneously moving both components.

FIGS. 5-7 illustrate another embodiment of a guidewire 100′. Guidewire 100′ is similar to guidewire 100 shown in FIGS. 1-4 in that it includes a proximal portion 102, a transition region 106, and a distal portion 104. Proximal portion 102 includes a hypotube 108. Distal portion 104 includes coil 110, tip 114, forming ribbon 140, and a distal tube 112′. Distal tube 112′ is similar to distal tube 112 shown in FIGS. 1-4, except that distal tube 112′ does not include a bore 116. Instead, distal tube 112′ includes a longitudinal cross-cut 117 through a center of distal tube 112′. Cross-cut 117 may be formed in distal tube 112′ in any way known to those of ordinary skill in the art. For example, a mold cavity may include a cross-cut shaped insert if distal tube 112′ is formed via casting or injected molding. Alternatively distal tube 112′ may be formed via extrusion with cross-cut 117 formed by the profile of a die in the extrusion head.

Core wire 118 includes a proximal section 120, a transition section 122, and a distal section 124, as in FIGS. 1-4. Core wire 118 is movable between a proximal position shown in FIGS. 5 and 7 and a distal position shown in FIG. 6. When core wire 118 is in the proximal position, cross-cut 117 of distal tube 112′ closes, as shown in FIG. 7. When core wire 118 is pushed distally, distal section 124 opens cross-cut 117 sufficiently to accept distal section 124 such that core wire 118 advances distally to add further support to distal portion 102 of guidewire 100′.

In one embodiment of practicing the disclosed method, guidewire 100 or 100′ is inserted into the vasculature with core wire 118 in the proximal position such that guidewire 118 can be advanced through the tortuous bends of the vasculature. Upon reaching a lesion, occlusion, or other impediment, core wire 118 is advanced distally such that distal section 124 is disposed within distal tube 112, 112′, thereby stiffening distal portion 104 for improved pushability.

While various embodiments of the present disclosure have been described above, it should be understood that they have been presented by way of illustration and example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment.

1. A guidewire comprising:
a proximal portion including a proximal hollow tube havinn a first longitudinal axis;
a distal portion having a second longitudinal axis and including a coil mounted around a distal tube, said coil coupled to said proximal hollow tube, wherein said distal tube is made of an elastomeric material; and
a core wire disposed within said proximal hollow tube, wherein said core wire is movable between a distal position wherein a distal end of said core wire is disposed a first distance from a distal end of said distal tube and a proximal position wherein the distal end of said core wire is disposed a second distance from the distal end of said distal tube, wherein said second distance is greater than said first distance,
wherein the first and second longitudinal axes arc coaxial when said core wire is disposed in the distal position and when said core wire is disposed in the proximal position.

2. The guidewire of claim 1, wherein when the core wire is in the distal position, a portion of the core wire is disposed within the distal tube.

3. The guidewire of claim 1, wherein when the core wire is in the proximal position, none of the core wire is disposed within the distal tube.

4. The guidewire of claim 1, wherein when the core wire is in the proximal position, a portion of the core wire is disposed within the distal tube.

5. The guidewire of claim 1, wherein said distal tube includes a bore sized and shaped to receive a distal section of said core wire.

6. The guidewire of claim 1, wherein said core wire includes a proximal section, a distal section, and a transition section disposed between said proximal section and said distal section.

7. The guidewire of claim 6, wherein said distal section has first diameter, said proximal section has a second diameter larger than the first diameter, and said transition section transitions from the second diameter to the first diameter.

8. The guidewire of claim 1, wherein the distal tube includes a cross-cut section such that the distal tube is closed when said core wire is in the proximal position and the core wire open the cross-cut when said core wire is in the distal position.

9. The guidewire of claim 1, wherein said proximal tube is a hypotube made from a material selected from the group consisting of stainless steel and cobalt chromium alloy.

10. The guidewire of claim 1, wherein said distal tube is made from a material selected from the group consisting of natural rubber, polyisoprene, butyl rubber (copolymer of isobutylene and isoprene, IIR), halogenated butyl rubbers (chloro butyl rubber: CIIR; bromo butyl rubber: BIIR), polybutadiene, styrene-butadiene rubber (copolymer of polystyrene and polybutadiene, SBR), nitrile rubber, (copolymer of polybutadiene and acrylonitrile, NBR), Therban® and Zetpol® hydrated nitrile rubbers (HNBR), Baypren® chloroprene rubber (CR), polychloroprene, neoprene, ethylene propylene rubber, ethylene propylene diene rubber, epichlorohydrin rubber (ECO), polyacrylic rubber (ACM, ABR), silicone rubber (SI, Q, VMQ), fluorosilicone rubber (FVMQ), fluoroelastomers, and perfluoroelastomers.

11. The guidewire of claim 1, further comprising a tip bonded to a distal end of the distal tube.

12. The guidewire of claim 11, further comprising a forming ribbon disposed within said coil, wherein a proximal portion of said forming ribbon is coupled to said proximal hollow tube and a distal portion of said forming ribbon is coupled to said coil and said tip.

13. A guidewire comprising:
a proximal portion including a proximal hollow tube having a first longitudinal axis;
a distal portion including a coil disposed around a distal tube, said coil being coupled to said proximal hollow tube and having a second longitudinal axis, wherein said distal tube is made of an elastomeric material and has a third longitudinal axis; and
a core wire disposed within said proximal hollow tube, wherein said core wire has a fourth longitudinal axis and is movable between a proximal position and a distal position, wherein said distal portion of the guidewire is more flexible when said core wire is in the proximal position than when said core wire is in the distal position,
wherein said first, second, third, and fourth longitudinal axes are coaxial when said core wire is in the distal position.

14-22. (canceled)

23. The guidewire of claim 13, wherein said distal tube includes a bore sized and shaped to receive a distal section of said core wire.

24. The guidewire of claim 13, wherein said core wire includes a proximal section, a distal section, and a transition section disposed between said proximal section and said distal section.

25. The guidewire of claim 24, wherein said distal section has first diameter, said proximal section has a second diameter larger than the first diameter, and said transition section transitions from the second diameter to the first diameter.

26. The guidewire of claim 13, wherein the distal tube includes a cross-cut section such that the distal tube is closed when said core wire is in the proximal position and the core wire open the cross-cut when said core wire is in the distal position.

27. The guidewire of claim 13, wherein said proximal tube is a hypotube made from a material selected from the group consisting of stainless steel and cobalt chromium alloy.

28. The guidewire of claim 13, wherein said distal tube is made from a material selected from the group consisting of natural rubber, polyisoprene, butyl rubber (copolymer of isobutylene and isoprene, IIR), halogenated butyl rubbers (chloro butyl rubber CIIR; bromo butyl rubber: BIIR), polybutadiene, styrene-butadiene rubber (copolymer of polystyrene and polybutadiene, SBR), nitrile rubber, (copolymer of polybutadiene and acrylonitrile, NBR), Therban® and Zetpol® hydrated nitrile rubbers (HNBR), Baypren® chloroprene rubber (CR), polychloroprene, neoprene, ethylene propylene rubber, ethylene propylene diene rubber, epichlorohydrin rubber (ECO), polyacrylic rubber (ACM, ABR), silicon rubber (SI. Q, VMQ), fluorosilicone rubber (FVMQ), fluoroelastomers, and perfluoroelastomers.

29. The guidewire of claim 13, further comprising a tip bonded to a distal end of the distal tube.

30. The guidewire of claim 29, further comprising a forming ribbon disposed within said coil, wherein a proximal portion of said forming ribbon is coupled to said proximal hollow tube and a distal portion of mid forming ribbon is coupled to said coil and said tip.
